(19) United States
(12) Patent Application Publication
(10) Pub. No.: US 2026/0243363 A1
(43) Pub. Date: Aug. 20, 2026

(54) HIGH SENSITIVITY PRESSURE RELIEF VALVE FOR WASTE GAS COLLECTION SYSTEMS

(71) Applicant: Watershed Geosynthetics, LLC, Alpharetta, GA (US)

(72) Inventor: Delaney LEWIS, Downsville, LA (US)

(73) Assignee: Watershed Geosynthetics, LLC, Alpharetta, GA (US)

(21) Appl. No.: 19/058,472

(22) Filed: Feb. 20, 2025

(51) Int. Cl. F16K 17/12; B09B 1/00; E02D 31/00

(52) U.S. Cl. CPC F16K 17/12; B09B 1/00; E02D 31/00

(57) ABSTRACT

A pressure relief valve for waste piles includes a housing and a waste gas conduit with an outlet positioned at least partly within the housing. A lightweight movable valve closure is provided for closing the outlet and uncovering the outlet, sensitive to very low gas pressures. The movable valve closure includes a lightweight, puck-shaped polymer foam body having a concave recess formed in an underside thereof and a neoprene rubber membrane covers the concave recess. The pressure relief valve is adapted to prevent a waste pile cover from ballooning in the event that a negative pressure pump stops operating.

### TECHNICAL FIELD

The present invention relates generally to landfills and, in particular, to a pressure relief valve for systems for collecting waste gas from a waste pile while minimizing the risk of dislodging a membrane positioned over the waste pile in the event of a power failure.

### BACKGROUND

Landfills are created incrementally and, as they grow, they do so in a largely linear path. They tend to start at one end of a site and as one initial block or area becomes full, another block or area of landfill is opened up next to it. Over time, the landfill grows much like adding fat/thick slices of a baguette back together to reassemble the baguette.

For many years, landfills were left uncovered. In more recent years, they have been covered with grass or dirt, both of which permit some or all of the gas created by the decomposition of the waste to escape to the atmosphere. It has become common to collect the landfill gas via deep wells connected to a large pump (fan or vacuum source) to pull the landfill gas from the landfill and to burn it at a flare on-site or nearby.

In more recent years, some landfills have begun to be capped or covered with an impermeable membrane. Given how the landfills grow and develop incrementally as described above, it often occurs that some parts of a landfill have an impermeable membrane, while other parts of the landfill do not. In these situations, when the gas collection system is offline (for instance, due to a power outage, mechanical failure, weather event, etc.), pressure differentials in the landfill and/or in the gas collection system can cause the impermeable membranes to “balloon”, destabilizing the installation of the membranes and damaging the landfill.

In the past, it has been known to use pumps, piping, and wellheads to extract the gases from the landfill and collect the same. Such wellheads are often spaced about one per acre in a grid pattern. Such systems of collecting the gases can be shut down by many factors, including power failures. To prevent the undesirable build-up of such gases in the event of non-operation of the extraction system, it has often been known to employ a grid pattern of vents spaced between the extraction wellheads, often at the same one per acre density.

As described in published U.S. patent application No. 20060034664, conventional gas extraction wells at landfills often involve deep wells attached to a network of pipes and a gas pump (blower) that applies vacuum (negative pressure) to extract the gas from the stored waste as the waste decomposes.

A prior art deep well arrangement according to the above published patent application is shown in FIG. 1. Landfill 1 containing waste W generates biogas (biogas flows shown by the arrows). Biogas is collected and extracted through a well 3. The well 3 includes a gas-collecting well screen 16 and a gas-impermeable conduit 17 linking the well screen to the surface to draw biogas from the wellhead to the surface. Overlaying the majority of the waste W is a gas-permeable layer 5. The term “wellhead” refers to a portion of the gas-extraction well from which gas can be extracted. The well often includes a section of pipe having slots or other gas-flow apertures cut in it, referred to as a “well screen”. Often, the well screen is also surrounded with gravel.

The gas-permeable layer is typically composed of a conductive porous matrix with gas flow paths. Often it is composed of rigid or semi-rigid particles of a large enough size to leave a significant void volume between particles. For instance, the gas-permeable layer may contain sand, gravel, wood chips, or shredded tires. Above the gas-permeable layer is a gas-containment layer 7. Biogas that rises from the landfill reaches the gas-permeable layer where it is trapped by the overlying gas-containment layer 7. The biogas migrates horizontally in the gas-permeable layer until it comes near to a well. Gas extraction from the well creates a vacuum that draws gas into the well. This vacuum draws biogas from the overlying gas-permeable layer down through the waste mass of the landfill to reach the well.

Beneath the gas-permeable high conductivity layer 5 through which a substantial fraction of the biogas from the gas-permeable layer passes as it travels to the gas-collection wellhead is an entrainment zone 9. On its passage through the waste 2, the gas from the gas-permeable layer mixes with biogas produced in the waste mass that has not gone through the gas-permeable layer. This helps to give a consistent content to the biogas that is withdrawn from the well. If gas is withdrawn directly from the gas-permeable conductive layer, the gas composition will vary more dramatically over time, sometimes containing a high air content and sometimes not. It is sometimes desirable to place an even more impermeable layer, such as geomembrane 15, directly over the zone of entrainment of gas from the permeable layer that is created by the deep well. Moreover, sometimes the entire landfill is covered with such a membrane.

The deep well design of FIG. 1 is designed to pull gas away from the surface to protect the membrane cover from being impacted with gas buildup that can create ballooning. Typically, the deep well has a diminishing radius (zone) of influence as a result of pressure loss through the length of the well collector pipes. The deep well vacuum pressure pulls both gas and leachate into the well. Leachate pumps are often required, resulting in more membrane penetrations. The membrane cover helps alleviate air intrusion issues—however, multiple penetrations typically are required at each collection point. Membrane penetrations around wellheads are very susceptible to rips and tears, and can result in either gas leaks or air intrusion into the waste. Another drawback to the deep well is that the deep well must be continually monitored and adjusted. Deep wells normally utilize an adjustable valve at each collection point to control pressures within the well to adjust the radius of influence, but have limited maximum radius of influence from the control valve.

FIG. 2 shows another prior art arrangement, this time showing a more shallow wellhead 26 used to withdraw near-surface or sub-surface gas from beneath a membrane M capping a waste W. The wellhead 26 is attached to an above-ground conduit by way of a vertical pipe.

FIG. 3 shows another prior art arrangement, this time depicting a landfill with multiple wellheads 30 used to withdraw near-surface or sub-surface gas from beneath the surface. The wellheads 30 are attached to an above-ground vent 31.

FIG. 4 shows another prior art arrangement similar to that in FIG. 2, this time showing a field of wellheads 40 spaced to extract the gases from a landfill and collect the same. Such wellheads are often spaced about one per acre.

One particularly troublesome problem with known prior art gas collection systems for use in landfills is that the systems typically use electrically-powered vacuum pumps to help draw off the waste gas. In the event of a power outage, such as often can occur due to lightning strikes, the waste gas can build up underneath a membrane in the landfill while the electric power is off, resulting in a ballooning effect on the membrane. This ballooning can damage the membrane or even cause it to slide off the side of a waste pile. Accordingly, a need exists for devices that can help keep the membrane intact without ballooning in the event of a power outage or failure of the vacuum pump. It is to the provision of such that the present invention is at least partially directed.

### SUMMARY OF THE INVENTION

Generally described, in a first example form the present invention relates to collection systems for collecting waste gas from a waste pile having an impermeable membrane covering some or substantially all of the waste pile and in particular relates to a pressure relief valve for such use. The pressure relief valve preferably includes a housing and a waste gas conduit with an outlet positioned at least partly within the housing. A movable valve closure is provided for closing the outlet and uncovering the outlet, sensitive to gas pressure. The movable valve closure includes a lightweight, puck-shaped body comprising a polymer foam and having a concave recess formed in an underside thereof. A membrane covers at least the concave recess formed in the underside of the puck-shaped body. Advantageously, the pressure relief valve is adapted to prevent the waste pile cover from ballooning in the event that the negative pressure pump stops operating.

Preferably, the polymer foam comprises polyethylene closed cell foam and the membrane comprises neoprene rubber. Optionally, the neoprene rubber membrane has a thickness of about 0.010 inches. Also optionally, the outlet conduit has an OD of between about 1 inch and 4 inches. In one example form, the outlet conduit has an OD of about 2 inches.

Advantageously, the pressure relief valve is operative to vent waste gas at pressures of between 0.1 inches and 30 inches of water column head. Also, the pressure relief valve can be operative to vent waste gas at pressures as low as between 0.1 inches and 1 inches of water column head.

In another example form, a pressure relief valve is provided for use with gas collection systems for collecting waste gas from a waste pile having a covering and a negative pressure pump. Preferably, the pressure relief valve includes a housing and a waste gas conduit with an outlet positioned at least partly within the housing. A movable valve closure is provided for closing/covering the outlet and opening/uncovering the outlet, sensitive to gas pressure. The movable valve closure includes a lightweight body and a flexible lower membrane for at times covering the outlet of the waste gas conduit. With this construction, the pressure relief valve is adapted to prevent the waste pile cover from ballooning in the event that the negative pressure pump stops operating and is sensitive to open in the presence of waste gas pressures as low as below about 1 inch of water column or even lower. Also, the pressure relief can even be operable to open in the presence of waste gas pressures as low as below about 0.1 inch of water column.

The specific techniques and structures employed to improve over the drawbacks of the prior systems and accomplish the advantages described herein will become apparent from the following detailed description of example embodiments and the appended drawings and claims.

### BRIEF DESCRIPTION OF THE DRAWING FIGURES

FIG. 1 is a schematic illustration of a prior art deep well arrangement for use at a landfill.

FIG. 2 shows another prior art arrangement, this time showing a more shallow wellhead used to withdraw near-surface or sub-surface gas from beneath a membrane capping a waste pile.

FIG. 3 shows another prior art arrangement, this time depicting a landfill with multiple wellheads used to withdraw near-surface or sub-surface gas from beneath the surface

FIG. 4 shows another prior art arrangement similar to that in FIG. 2, this time showing a field of wellheads spaced to extract the gases from a landfill and collect the same.

FIG. 5 is a schematic, elevation illustration of a pressure relief valve according to a first example embodiment, such as for venting waste gas from a waste pile having an impermeable membrane.

FIG. 6A is a schematic, perspective illustration of a portion of the pressure relief valve of FIG. 5, with some elements removed for clarity of illustration, and showing both a movable valve closure and a waste gas outlet conduit portion of the pressure relief valve.

FIG. 6B is a schematic, perspective illustration of a portion of the pressure relief valve of FIG. 5, with some elements (including the movable valve closure) removed for clarity of illustration, and showing a waste gas outlet conduit portion of the pressure relief valve.

FIG. 7A is a schematic, sectional illustration of a portion of the pressure relief valve of FIG. 5, with some elements removed for clarity of illustration, and showing both a movable valve closure and a waste gas outlet conduit portion of the pressure relief valve, and depicting the valve in a closed configuration.

FIG. 7B is a schematic, sectional illustration of a portion of the pressure relief valve of FIG. 5, with some elements removed for clarity of illustration, and showing both a movable valve closure and a waste gas outlet conduit portion of the pressure relief valve, and depicting the valve in an opened configuration.

FIGS. 8A-8D are schematic illustrations of optional shapes for a body portion of the movable valve closure of FIG. 6A.

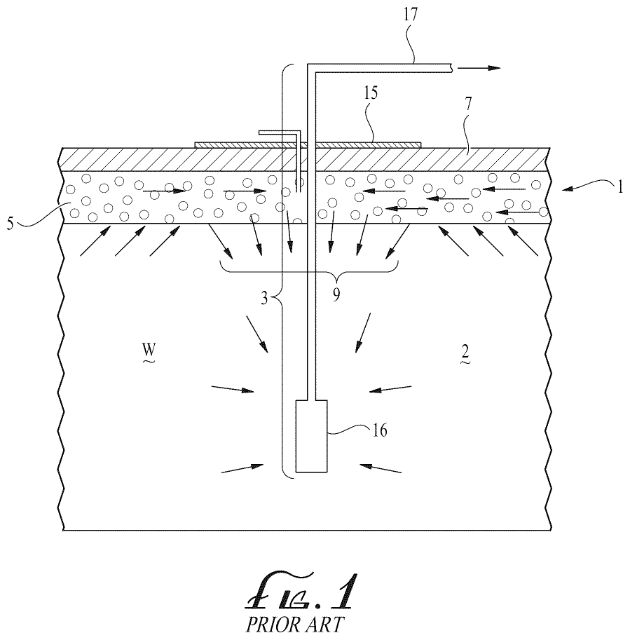

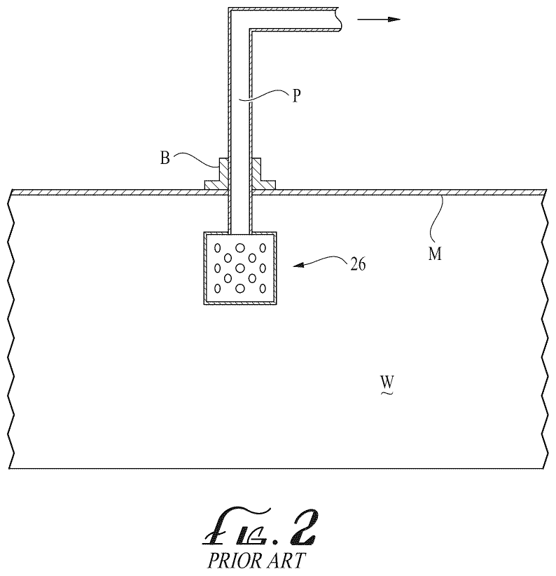

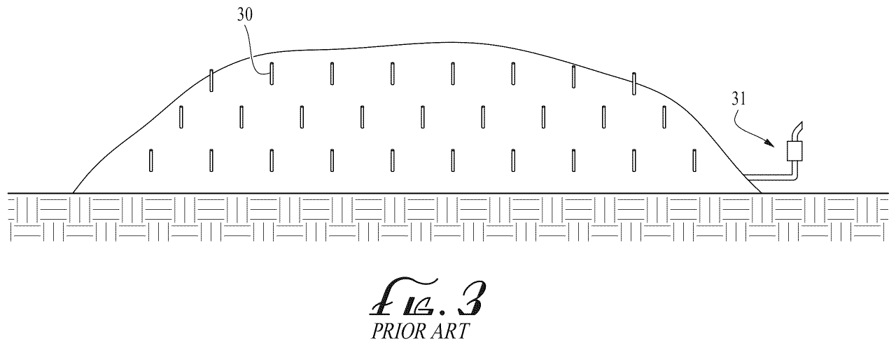

### DETAILED DESCRIPTION OF EXAMPLE EMBODIMENTS

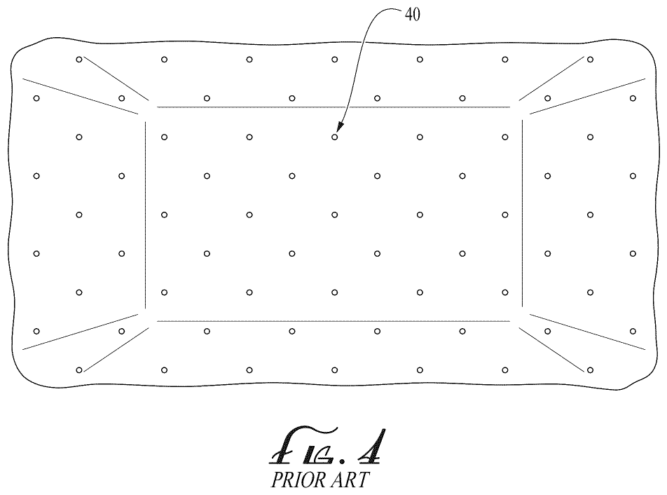

Referring now in detail to FIGS. 5-8D, in example forms the present invention relates to pressure relief valves for gas collection systems for collecting waste gas from waste piles, such as from landfills. FIG. 5 shows a pressure relief valve 500 for venting waste gas from a waste pile having a covering, such as an impermeable membrane, which covers part of or substantially all of the waste pile. The pressure relief valve 500 includes a bottom portion 510 which is generally bottle-shaped. A lowermost portion of the bottle-shaped bottom portion 510 can be coupled to the gas collection grid of the waste site using adapter 550. The lower portion 510 includes one or more pressure relief apertures, such as pressure relief apertures 511 and 512. These pressure relief apertures operate to allow waste gas which is selectively vented through the internal valve to escape through the lower portion housing 510. These apertures 511, 512 can be slits, perforations or holes.

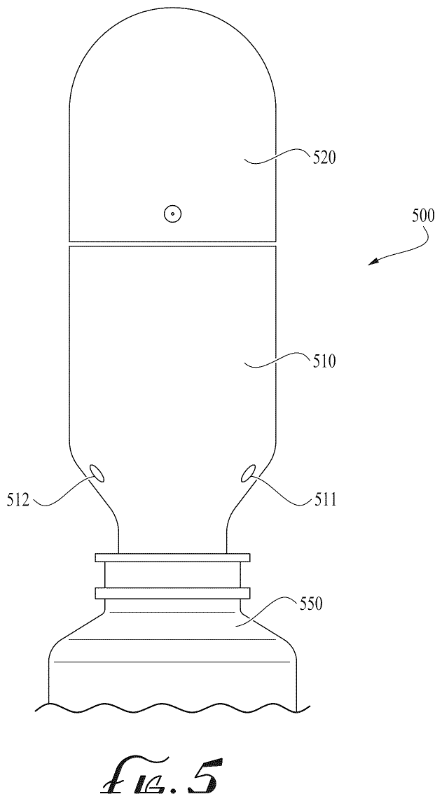

The pressure relief valve 500 also includes an upper portion 520. The upper portion 520 is removably secured to the lower portion 510 and can be removed from the lower portion 510 as depicted in FIGS. 6A and 6B. Positioned within the lower portion 510 is a gas outlet conduit 560 having an annular surface 561 at its upper end. The gas outlet conduit 560 can be placed in fluid communication with the waste gas collection grid of the waste site. As shown in FIG. 6B, the gas outlet conduit 560 can be in the form of a vertical pipe 562 with an upper outlet aperture 564 formed therein. The outlet conduit 560 is capped by a movable body or valve closure 570 (see FIG. 6A). The movable valve closure 570 includes a generally puck-shaped main body portion 571 and a thin neoprene rubber membrane or covering 572. In one example form, the neoprene rubber covering 572 has a thickness of about 0.010 inches, but other thickness can be employed. As shown in FIG. 6A, the neoprene rubber membrane covers the sides of the puck 571 and slightly overlaps the top of the puck 571. The rubber membrane 572 also covers the underside of the puck-shaped body portion 571.

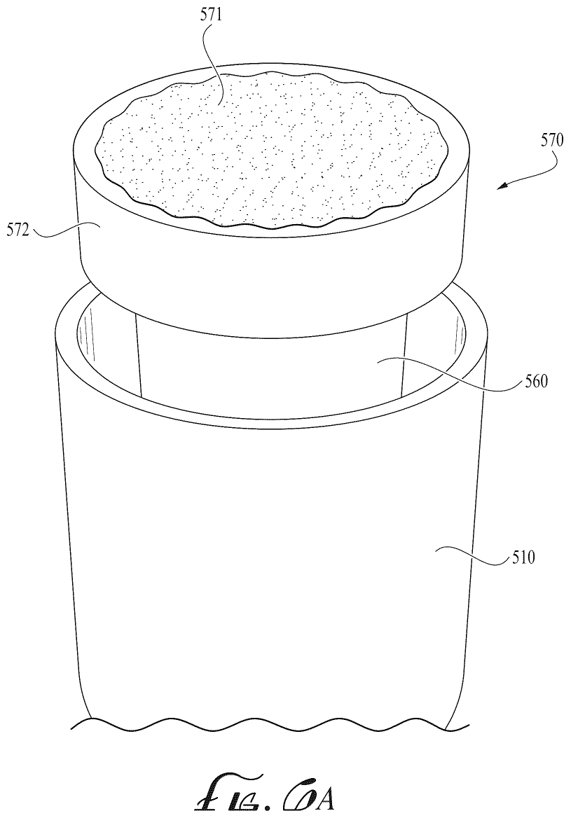

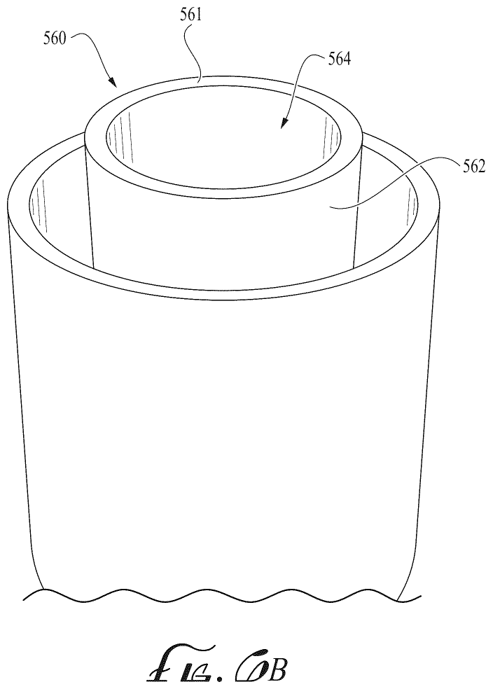

As shown in FIG. 7A, the covering or sleeve 572 includes a lower portion 572a that spans the bottom of the puck 571. It also includes a side portion 572b that covers the sides of the puck 571. The covering or sleeve 572 further includes an upper portion 572c that overlies an upper part of the puck 571 along the edges thereof. It should be noted that the figures are not drawn to scale and that the thickness of the covering or sleeve 572 if drawn to scale would appear much thinner than what is shown in FIGS. 7A and 7B.

As shown in FIGS. 7A and 7B, an underside portion 571A of the puck 572 includes a concave recess 573, while the upper side 571b of the puck 571 is substantially flat. The concave recess 573 reduces the mass/weight of the puck, while also providing some clearance to allow the membrane some space to be deformed slightly as it seals against the upper annular surface 565 of the vertical pipe 562. In this regard the recess facilitates the lower portion 572a of the neoprene rubber membrane or covering to conform to and seal against the annular surface 561 of the outlet conduit.

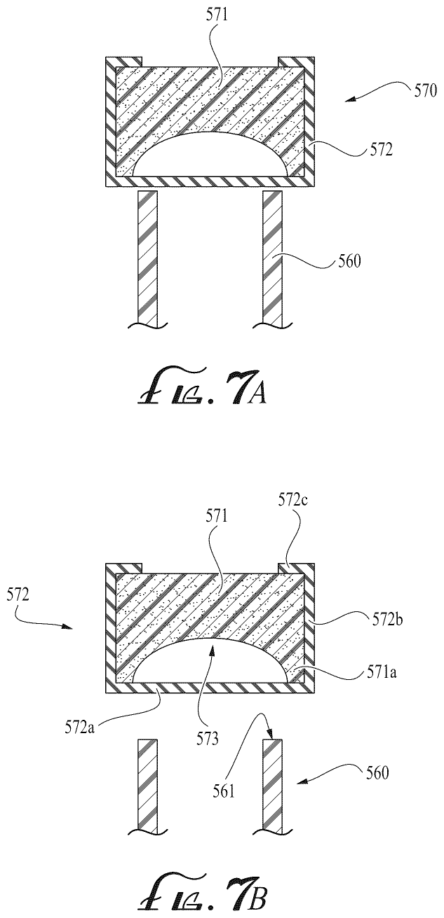

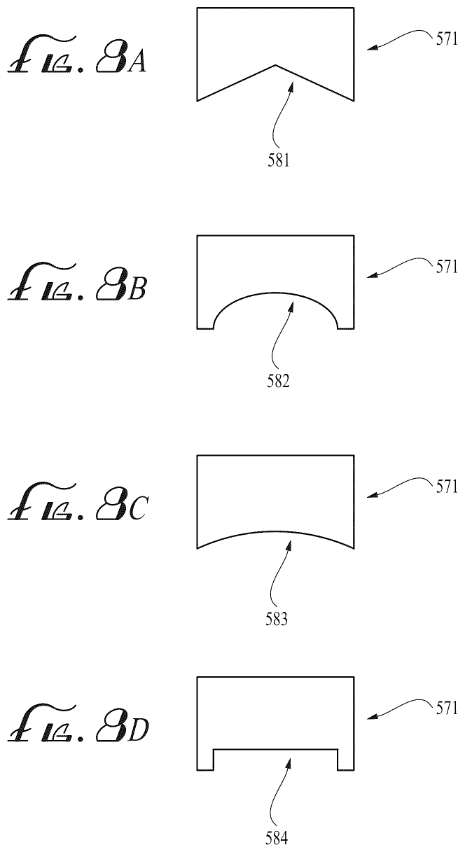

While FIGS. 7A and 7B depict an elliptical recess of moderate depth, it will be appreciated that other shapes can be employed. For example, as schematically depicted in FIGS. 8A-8D, conical, elliptical, shallow spherical, or cylindrical recesses can be employed.

The movable valve closure 570 preferably is made of a light-weight closed cell foam, together with a neoprene rubber material. Polyethylene closed cell foam has been found to work well for the puck body 571. The materials are two separate components that are assembled to create a final product. In one example form, the foam puck body 571 has an OD of about 4″ and a thickness of about 1″. The lower side of the foam puck body 571 preferably has a concave shape and has a flat surface on the other side. Optionally, the neoprene rubber sleeve 572 has a slightly smaller diameter than the foam puck body 571 so that it fits tightly onto the foam puck body 571. In one example form, the neoprene sleeve 572 has a thickness of about 0.010 inches.

The neoprene sleeve 572 is installed across the foam puck body 571 so that the lower membrane is stretched across the concave side of the foam. This creates a surface area that is very flexible and allows the neoprene to fluctuate in and out (because of the concave shape of the underlying foam) to seal and release against the valve stem with the demands of very slight pressure differentials.

In one example form, the gas outlet conduit 560 optionally has an OD of between about 1 inch and 4 inches. Preferably, the OD is about 2 inches (and thus the ID would be less than 2 inches). Optionally, the annular surface 561 can be beveled to reduce surface tension or stickiness between the annular surface and the neoprene rubber membrane 572. In this regard, it may be advisable to start the bevel from the outside of the conduit, to keep from increasing the surface area of the conduit opening by enlarging the mouth of the opening.

The total unit weight of the example puck 571 and sleeve/covering 572 is 12 g, which allows for a pressure relief valve of considerable sensitivity to very low gas pressures. Indeed, the novel pressure relief valve according to the present invention has been found to be opened with just a fraction of an inch of water column pressure.

In theory, the sensitivity (valve opening pressure) is related to the weight of the movable valve body 570 divided by the surface area of the outlet opening 564 of the gas outlet conduit 560. The opening pressure is determined by the following theoretical formula,

P=W/Aₒ

Where P is the waste gas pressure that will open the valve, W is the weight of the movable valve body 570, and Aₒ is the surface area of the outlet opening 564 of the gas outlet conduit 560. Of course, that theoretical opening pressure is influenced by other factors, including surface tension (stickiness) of the neoprene rubber membrane 572 to the annular surface 564 of the gas conduit 560. Reducing the total area of the annular surface 564 can lower the opening pressure.

This valve design is designed to meet the demands presented by very low confining weights associated with interim membrane cover systems. In other words, in some waste sites, an interim (temporary) cover is placed over the waste pile and the interim covers are often rather lightweight or flimsy and are easily dislodged by waste gas pressures that are rather low. The present invention helps to keep those interim covers in place over a waste pile, even when the power goes off and the vacuum systems stop drawing the waste gas out of the collection grid. Such pressure relief valves can be provided to prevent a membrane from ballooning in the event that a negative pressure pump stops operating for a time. In example forms, the base portion 654 and cap 655 comprise a generally cylindrical profile. However, the base portion and cap may comprise other geometric profiles, such as for example polygonal or elliptical profiles, of various dimensions, and it is to be understood that the base portion and cap are not limited to any specific profile or size.

Preferably, the pressure relief valves are adapted to vent waste gas at pressures of between about 0.1 inch and 30 inches of water column head. The novel pressure relief valve design disclosed herein has been found to be particularly sensitive and even is operative to open at waste gas pressures of well below 1 inch of water column head, a sensitivity heretofore unknown in the industry. Indeed, novel pressure relief valve design disclosed herein has been found to be particularly sensitive and even is operative to open at waste gas pressures of below 0.1 inch of water column head.

Advantageously, the novel pressure relief valves disclosed herein also operate to prevent back flow (in the opposite direction of the relief provided), even rather slight back flows.

It is to be understood that this invention is not limited to the specific devices, methods, conditions, or parameters of the example embodiments described and/or shown herein, and that the terminology used herein is for the purpose of describing particular embodiments by way of example only. Thus, the terminology is intended to be broadly construed and is not intended to be unnecessarily limiting of the claimed invention. For example, as used in the specification including the appended claims, the singular forms “a,” “an,” and “the” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

While the claimed invention has been shown and described in example forms, it will be apparent to those skilled in the art that many modifications, additions, and deletions can be made therein without departing from the spirit and scope of the invention as defined by the following claims.

What is claimed is:

1. A pressure relief valve for use with a gas collection system for collecting waste gas from a waste pile having a covering and a negative pressure pump, the pressure relief valve comprising:
A housing;
A waste gas conduit with an outlet and positioned at least partly within the housing;
A movable valve closure for closing the outlet and uncovering the outlet, sensitive to gas pressure and comprising a lightweight puck-shaped body comprising a polymer foam and having a concave recess formed in an underside thereof, and a membrane covering at least the concave recess formed in the underside of the puck-shaped body; wherein the pressure relief valve is adapted to prevent the waste pile covering from ballooning in the event that the negative pressure pump stops operating.

2. A pressure relief valve as claimed in claim 1 wherein the polymer foam comprises polyethylene closed cell foam.

3. A pressure relief valve as claimed in claim 1 wherein membrane comprises neoprene rubber.

4. A pressure relief valve as claimed in claim 1 wherein the neoprene rubber membrane has a thickness of about 0.010 inches.

5. A pressure relief valve as claimed in claim 1 wherein the outlet conduit has a OD of between about 1 inch and 4 inches.

6. A pressure relief valve as claimed in claim 5 wherein the outlet conduit has a OD of between about 2 inches.

7. A pressure relief valve as claimed in claim 1 wherein the pressure relief valves is operative to vent waste gas at pressures of between 0.1 inches and 30 inches of water column head.

8. A pressure relief valve as claimed in claim 1 wherein the pressure relief valves is operative to vent waste gas at pressures as low as between 0.1 inches and 1 inches of water column head.

9. A pressure relief valve for use with a gas collection system for collecting waste gas from a waste pile having a covering and a negative pressure pump, the pressure relief valve comprising:
A housing;
A waste gas conduit with an outlet and positioned at least partly within the housing;
A movable valve closure for closing the outlet and uncovering the outlet, sensitive to gas pressure and comprising a lightweight body, and a lower flexible membrane for at times covering the outlet of the waste gas conduit; wherein the pressure relief valve is adapted to prevent the waste pile covering from ballooning in the event that the negative pressure pump stops operating and is sensitive to open in the presence of waste gas pressures as low as below about 1 inch of water column.

10. A pressure relief valve as claimed in claim 9 wherein the pressure relief valve is operable to open in the presence of waste gas pressures as low as below about 0.1 inch of water column.
